(19) United States
(12) Patent Application Publication
(10) Pub. No.: US 2002/0042069 A1
(43) Pub. Date: Apr. 11, 2002

(54) Long-length oligonucleotide microarrays

(76) Inventors: Vickesh E. Myer, Harvard, MA (US); David G. Statham, Arlington, MA (US); Thomas L. Volkert, Cambridge, MA (US); Christopher J. Wilson, Cambridge, MA (US)

Correspondence Address: Corning Incorporated, SP-TI-03-1, Corning, NY, 14830, US

(21) Appl. No.: 09/859,752

(22) Filed: May 17, 2001

Related U.S. Application Data
(60) Provisional application No. 60/204,846, filed on May 17, 2000.

(51) Int. Cl. C12Q 1/68; C12M 1/34

(52) U.S. Cl. 435/6; 435/287.2

(57) ABSTRACT

A method for producing a DNA microarray for biological assays comprising a plurality of biosites located on a solid substrate. Each biosite has at least one set of chemically presynthesized, oligonucleotides of medium to long lengths attached to the substrate by predominantly non-covalent means, such as electrostatic, hydrophobic, hydrogen bonding, van der waal forces, etc. The oligonucleotides exhibit no predetermined spatial orientation on the substrate surface. The oligonucleotides are chemically unmodified having a length of about 30 bases or longer. Preferably, the oligonucleotides are from about 45 bases to 85 bases, but some embodiments may include very long oligonucleotides of over 100 bases.

### CLAIM OF PRIORITY

This Application claims priority of U.S. Provisional Application No. 60/204,846, filed on May 17, 2000, the content of which are incorporated by reference.

### FIELD OF THE INVENTION

The invention relates to the field of molecular biology, and more particularly to the field of assays, or “DNA-chip” technology, that involve nucleic acid hybridization. The invention provides a method for making and using array-based nucleic acid hybridization substrates.

### BACKGROUND

DNA-microarrays provide a means to quantify tens of thousands of discrete genetic sequences in a single assay. As innovative tools, DNA microarrays have a number of various applications, including the parallel analysis of gene transcription profiles en masse, DNA polymorphisms, and other DNA or RNA hybridization assays. Among the most widespread uses of DNA microarrays is expression profiling, which has found many uses including discovery of gene functions, drug evaluation, pathway dissection, and classification of clinical samples. Based on the use of such arrays of nucleic acid probes on solid surfaces, large-scale mutational analysis and the analysis of gene expression are becoming a reality. The key to these advances has been the development of immobilization chemistry for the spatially resolved attachment of DNA probes to a solid support surface, so as to form the desired microarrays.

Recent research in the microarray field has concentrated on the development covalent coupling of relatively short oligonucleotide probes to planar surfaces. Such covalent coupling requires activation of the underlying planar surface with cross-linking reagents and/or modifications of the DNA molecule with a reactive group. Alternatives using non-covalent approaches have not been particularly successful. Some have suggested that DNA probe molecules attached to the surface of a substrate by multiple constraining contacts would serve as poor hybridization probes, because of the loss of configurational freedom and the attendant loss of capacity to form double stranded bonds between targets and probes. Non-covalently attached DNA molecules would be susceptible to removal from the substrate surface, if a few contacts were made between the nucleic probe and surface. Therefore, the DNA (oligo) microarray field has focused most of its attention on covalent attachment strategies.

A critical, but often time-consuming and costly step in the production of DNA microarrays is the up-front procurement and selection of high quality DNA content. Two major types of platforms for manufacture of high-density microarrays have been developed. The first platform involves relatively short (≦25 bases) oligonucleotides made by a photolithographic process similar to the manufacture of computer chips. This type of fabrication is often a multi-stepped process that involves in-situ synthesis of oligonucleotides on a solid substrate, such as a slide. The second platform uses robotic deposition or “spotting” of DNA molecules onto a specially coated substrate. Spotted arrays are commonly referred to as “cDNA microarrays,” although clones, PCR amplified products, or oligonucleotides (pre-synthesized) can all be spotted onto a specially coated substrate. To provide the linkage and spacer elements for attaching an oligonucleotide to a slide surface, a pre-synthesized oligonucleotide is usually chemically modified by the addition of a functional group to its 5′ or 3′ end.

Each of these two approaches for microarray designs possesses a number of virtues. For example, on one hand, arrays that use PCR-product sequences tend typically to exhibit a better signal than devices with printed oligonucleotides. Oligonucleotides arrays, on the other hand, offer greater specificity than cDNAs, including PCR products, having the capability to distinguish single nucleotide polymorphisms and discern splice variants. In other words, oligonucleotide arrays are independent of cDNA templates, and their relatively short length avoids repetitive homologues of PCR amplified nucleic acid sequences, which may lead to problems of cross hybridization and high background signal.

More importantly, however, are the shortcomings of each platform. Sequencing efforts have greatly enlarged the bank of useful information available for microarray products, yet, for these diverse products the potential has not been realized using PCR amplified DNA. PCR, while useful for producing sizeable quantities of genetic sequences, is nonetheless DNA template dependent, thus subject to limitations in the potential variations of genetic expression. PCR forces an array manufacturer to select DNA content based on publicly or privately available DNA in the form of cDNA clones, genomic DNA primers, or PCR amplified templates. Moreover, PCR can become a bottleneck during product development or manufacture when templates sources, such as EST clones, full-length cDNA, or other DNAs, becomes unavailable. For example, microarray product concepts such as toxicology arrays or bacterial genome arrays without template sources cannot be manufactured until original template sources are identified or made.

Relatively short oligonucleotides (≦25-mer) on arrays, in contrast, do not provide the requisite level of signal for improved imaging, nor do they bind well to sample target nucleic sequences during hybridization because of steric problems associated with their short lengths and the secondary structures of labeled targets. Also, even presynthesized oligonucleotides that are chemically modified have a drawback. These oligonucleotides, as a consequence of being chemically modified, require special surfaces for attachment, and may lessen performance and specificity. The modified oligonucleotides tend to function less naturalistically than unmodified oligonucleotides when binding to target nucleic sequences. Moreover, in both systems, creation of a new array design is relatively inconvenient and/or expensive, requiring either a new set of masks for photolithography, or new samples to deposit for spotted cDNA arrays. Flexibility to create new arrays is becoming increasingly important as more genomes are sequenced and more applications for microarrays are described.

Hence, an invention which can combine the advantages of both PCR and oligonucleotide microarrays, and provide a viable, non-covalent adsorption to a planar substrate would likely receive a warm welcome from workers in the biological, medical, or pharmaceutical fields. The present invention is intended to meet such a need. The present invention provides a design for and a method of making DNA microarrays that employ sets of oligonucleotides having medium to long sequences (≧26 bases).

### SUMMARY OF THE INVENTION

The present invention in one aspect relates to a method for producing a microarray using chemically synthesized oligonucleotides in place of enzymatically amplified DNA. The method comprises, in part, providing at least one chemically synthesized, single-stranded oligonucleotide having a length of at least about 30 bases or longer, affixing the oligonucleotide to a substrate by a non-covalent means (e.g., electrostatic, hydrophobic, van der waal force, etc.), such that the oligonucleotide exhibits no predetermined orientation upon the substrate. The method preferably uses oligonucleotides that are not chemically modified. The present invention also relates to the resultant array that comprises single-stranded, chemically synthesized oligonucleotides of a length of about 30 bases or longer, attached by non-covalent means to a substrate, where the oligonucleotides are not oriented in a specific predetermined fashion. In some embodiments, preferably, the oligonucleotides have a length of about 45 bases, and more preferably over about 80 bases in length.

### BRIEF DESCRIPTION OF FIGURES

FIG. 1 is a schematic representation of a typical PCR-product sequence that is bound to a substrate and undergoing hybridization with a target sample nucleic sequence.

FIG. 2 is a schematic representation of a typical short oligonucleotide (≦25-mer), with a target sample nucleic sequence floating in solution above it.

FIGS. 3A and 3B are schematic representations of other embodiments of the present invention. FIG. 3A shows a long (˜80-mer to ˜100-mer) or very long oligonucleotide (≧100-mer) attached to a substrate, and undergoing hybridization with a target sample nucleic sequence. FIG. 3B shows a dendrimer having a number of medium-length oligonucleotides, to which target sample nucleic sequences can hybridize.

FIG. 4 shows a hybridization image of an array with PCR-product, 100-mer, 125-mer, and 150-mer sequences.

FIG. 5 shows a representative hybridization comparing long oligonucleotides and PCR-products printed on a GAPS slide.

FIG. 6 shows a scatter plot in Cyanine 3 of extracted intensity values from each spot of five the, where relative fluorescence units (RFUs) from the oligonucleotides are plotted along the x-axis and RFUs from the PCR-product are plotted along the y-axis.

FIG. 7 shows a scatter plot in Cyanine 5 of extracted intensity values from each spot of five the, where RFUs from the oligonucleotides are plotted along the x-axis and RFUs from the PCR-product are plotted along the y-axis.

FIG. 8 is a series of charts that plot relative fluorescence values from each of four hybridizations.

### DETAILED DESCRIPTION OF THE INVENTION

Use of oligonucleotides and PCR-products form the basis for two currently available methods used for detecting genetic polymorphisms and other types of nucleic assays. The present invention proposes to combine the best attributes of each of these two methods in a design and method of making a DNA array. One can significantly reduce the complexities of cDNA based arrays if chemically synthesized oligonucleotides of medium to long length are used as the probe material on DNA arrays, instead of an enzymatically produced large DNA fragment. Through proper definition of the parameters used in selecting oligonucleotides and the DNA sequence database, one also expects that an oligonucleotide based DNA array platform will deliver better performance and specificity than a cDNA based array.

The present invention provides methods for the manufacture of array devices and methods for use in the detection and/or isolation of nucleic acids. The devices and methods according to the invention employ predominantly non-covalent means of immobilizing nucleic acid or oligonucleotide probes. Increasing the size or length of an oligonucleotide will be useful for DNA hybridization assays when printed on solid or semi-solid, substrate surfaces. PCR typically produces DNA molecules greater than 200 base pairs, and it was thought that a DNA molecule smaller than this would not perform well in DNA hybridization assays. Herein, we demonstrate that presynthesized, non-modified, medium to long length oligonucleotides are useful for gene expression analysis. Unlike PCR product, oligonucleotide DNA is single stranded when printed to a substrate surface. Single stranded array probes should hybridize more effectively to the solution target because there is no internal competition for hybridization.

An oligonucleotide of any length will bind efficiently to a coated slide surface. As the length of an oligonucleotide increases, more regions on the probe oligonucleotide become available for hybridization. Theoretically, the longer the oligonucleotide is, the better target nucleic sequences will hybridize to it. Thus, an oligonucelotide with more hybridization binding sites is preferred over a shorter one. Unfortunately thus far, due to limitations of synthesis chemistry, synthesis of oligonucleotides that have a length greater than about 30-45-60 bases has been rather complex. And, preparation of oligonucleotides that have a length greater than about 80 bases is at the present rather difficult and inefficient, with a theoretical yield of full-length oligonucleotides at 20% or less. To overcome the synthesis limitations for extra-long oligonucleotides, to address steric concerns, and to improve the practicality of long oligonucleotides, the present invention has been developed.

The present invention provides a means of making a DNA array that employs a predominately non-covalent attachment mechanism without sacrificing probe stability or stringency. Since no oligonucleotide modification is required, this feature imparts a much easier way of manufacturing oligonucleotide DNA arrays. Further, oligonucleotides can be and are randomly anchored to a substrate surface, thus no bias exists between 5′ or 3′ ends when hybridizing to a target, which can also avoid steric effects at the surface. This virtue is a significant advantage over current, covalent attachment schemes that employ either 5′ or 3′ terminal strand attachment because they are hindred sterically from achieving good, unbiased hybridization. See for comparison, a paper by T. R. Hughes et al., entitled “Expression Profiling Using Microarrays Fabricated by an Ink-jet Oligonucleotide Synthesizer,” in NATURE BIOTECHNOLOGY, Vol. 19, pp. 342-347, April 2001, the contents of which are incorporated by reference herein. Moreover, by means of selecting an optimal oligonucleotide length (e.g., ˜65, ˜70, ˜75, or ˜85 bases) a balance between probe retention and hybridization maybe struck.

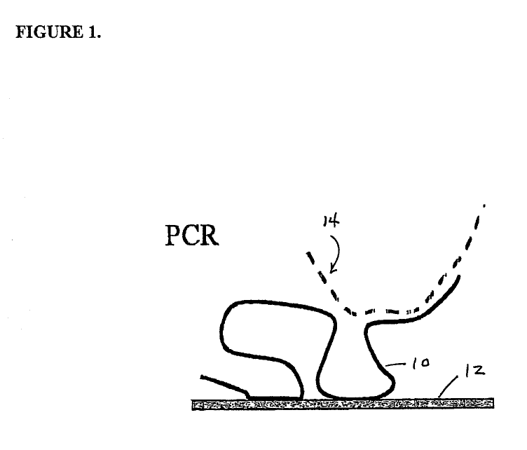

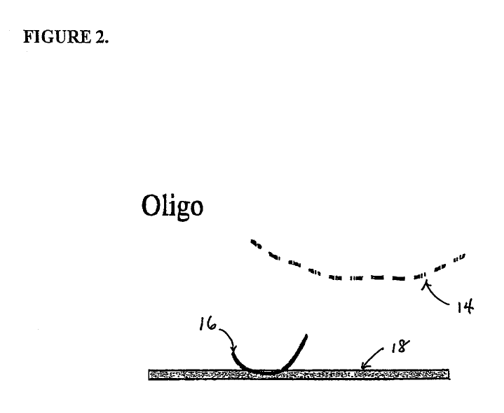

FIG. 1 shows a schematic representation of a typical PCR product (cDNA) 10 bound to a surface 12 and undergoing hybridization with a target nucleic sequence 14. The length of PCR products contributes to their relative stability of attachment, while providing good hybridization sites for the target. A long strand of PCR-product has multiple regions available for hybridization and regions that anchor itself to the substrate. In comparison, FIG. 2 shows a short length oligonucleotide structure 16, of the kind predominately used in current microarray designs, adsorbed to a substrate 18, attempting to hybridize with a target sequence 14. Short length oligonucleotides cannot bind as well as longer oligonucleotide sequences with long target sequences. The inherent short length of this kind of oligonucleotide limits its hybridization effectiveness. As one can see from FIG. 2, short oligonucleotides are not able to bind to gama-amino-propyl-silane (GAPS) coated substrates as efficiently as PCR-product sequences and still have enough sequence available for hybridization. The oligonucleotide strand is limit as to good potential hybridization sites. In contrast, the present invention has a greater specificity and stringency of hybridization, since a long oligonucleotide overcomes this problem and functions in a manner similar to PCR product.

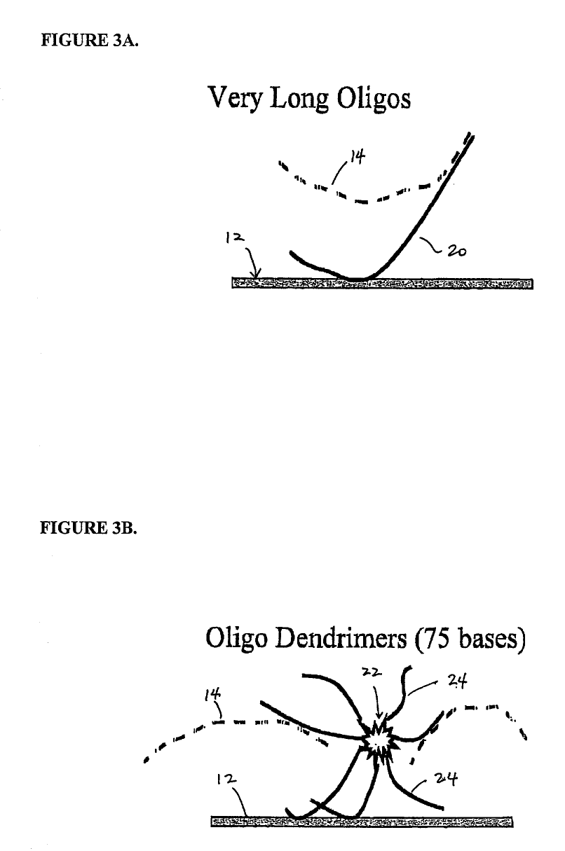

FIGS. 3A and 3B are schematic representations of other embodiments of the present invention. FIG. 3A illustrates a relatively long length oligonucleotide 20 attached to a substrate 12, and is undergoing hybridization with a target nucleic sequence 14. For purposes of the present invention, the synthesized oligonucleotides have a length of about 30 bases or greater, preferably about 40-45 bases or greater, and more preferably about 70-80 bases and longer (e.g., ˜100-mer, ˜125-mer, ˜150-mer, ˜180-mer). An upper limit of length is only limited by available current technology in producing high quality long oligonucleotides. FIG. 3B shows a multi-armed molecule, dendrimer 22, having attached to it a number of oligonucleotides 24 to which target nucleic sequences 14 may hybridize. Although oligonucleotide length is not a limiting factor, preferably the length for the dendrimer embodiment is in the range of about 50 to 85 bases, which behaves like a larger PCR product. Oligonucleotides that are about 50-60-75-80-100-150-180 bases, etc. or longer can perform as well as PCR products in detecting low level gene expression. In each of the aforementioned embodiments of the present invention, a fairly long length of oligonucleotide is exposed as potential hybridizing sites, while the entire oligonucleic structure or network is adhered securely, under common hybridization conditions, to the substrate surface by predominately non-covalent means.

Preferably, while performing hybridization assays with the present invention, each target nucleic sequence is labeled with more than one dye label moiety. In contrast to some other techniques that require fragmented, short target sequences for steric reasons, a microarray according to an embodiment of the present invention can make use of full length, multi-labeled targets, which increases the hybridization signal performance with respect to background fluorescence. The capability of increasing the number of dye markers incorporated per strand of target molecule at each binding site augments signal for each hybridized, individual, oligonucleotide strand, and the overall signal emitted from each biosite on the array. This phenomenon shows not only a better performance in respect to background fluorescence, but also a better compatibility in hybridization between a probe and target. The use of medium to long length oligonucleotides (≧30-45 bases) promotes the compatibility of probes to hybridize with targets. Oligonucleotides of medium to long lengths make it possible to use higher stringency in hybridization, which disrupts or reduces the formation of secondary structures in labeled target sequences, thereby enhancing affinity and hybridization between an oligonucleotide strand and labeled target. Further, the non-covalent attachment—as contrasted with current covalent attachment schemes—enables, the ologonucleotides to be randomly attached, without predetermined spatial orientation, to the substrate surface. This phenomenon, as mentioned before, reduces or eliminates bias between 5′ or 3′ ends when hybridizing to a target, thus avoiding steric effects at the surface.

Another virtue of the present invention relates to the ease of manufacture and design of microarrays. Synthesized sequences are liberated from dependence on cDNA templates. If one were to use chemically synthesized oligonucleotides, rather than PCR products, every conceivable DNA sequence would become available, and with greater specificity. Subject only to the sophistication of oligonucleotide-selection computer software, one can design virtually any gene sequence or combination of specific sites on an oligonucleotide strand, and avoid repetitive homologues which may lead to problems of cross hybridization and high background signal. Furthermore, the invention demonstrates that the length of an oligonucleotide, or the number of nucleotides, is critical for successful microarray hybridization experiments. Also, no oligonucleotide modification is required according to the present invention, in contrast to covalent methods.

### DEFINITIONS

Unless defined otherwise, all technical and scientific terms used herein have the meaning commonly understood by a person skilled in the art to which this invention relates. As used herein, the following terms have the meanings ascribed to them unless specified otherwise.

The term “array” or “microarray” or “DNA array” or “nucleic acid array” or “biochip” as used herein means a plurality of probe elements, each probe element comprising a defined amount of one or more nucleic acid or polypeptide molecules or targets, immobilized (including non-covalent associations, as described herein) to a solid surface or substrate.

The term “biosite” as used herein means a discrete area, spot or site on the active surface of an array, or base material, comprising at least one kind of predominantly non-covalent immobilized probe.

“Complementary” nucleic acid sequences are nucleotides on opposite strands that would normally base pair with each other.

A “nucleic acid target” can be a chromosome or any portion thereof, or can be a recombinant nucleic acid molecule, such as a plasmid, oligonucleotide, or other nucleic acid fragment, and may be naturally occurring or synthetic. The target length is not critical provided that the target is sufficiently long to complement the probe, as described herein. When the target is DNA, it is understood that the DNA is provided for use in the method in a partially denatured or single stranded form, capable of hybridizing to a single-stranded oligonucleotide probe.

The term “solid substrate” or “substrate surface” as used in this application is a solid or “semi-solid” material, which can form a solid support for the array device of the invention. The substrate surface can be selected from a variety of materials including, for example, polyvinyl, polystyrene, polypropylene, polyester, other plastics, glass, SiO₂, other silanes, hydrogels, gold or platinum, and the like.

The terms “hybridizing specifically to” and “specific hybridization” and “selectively hybridize to,” as used herein refer to the binding, duplexing, or hybridizing of a nucleic acid molecule preferentially to a particular nucleotide sequence under stringent conditions. The term “stringent conditions” refers to conditions under which a probe will hybridize preferentially to its target sequence, and to a lesser extent to, or nor at all to, other sequences. A “stringent hybridization” and “stringent hybridization wash conditions” in the context of nucleic acid hybridization are sequence dependent, and are different under different environmental parameters.

### EXPERIMENTS

Arrays were fabricated using oligonucleotides of varying sizes and purity using a gamma-amino-propyl-silane (GAPS) coated slide. The performance of oligonucleotides with a length great than 30-mer was found to be comparable to PCR product on GAPS slides after UV cross-linking. Both sensitivity and dynamic range on oligonucleotide arrays with oligonucleotide-lengths greater than 30-mer are comparable to cDNA targets. Differential gene expression was observed for all oligonucleotides, including 30-mer. We were able to confirm the expression level of differentially expressed gene with a real-time PCR method. Oligonucleotides from about 30 to about 80 nucleotide length can offer another option for fabricating DNA microarray on Corning™ GAPS slide without compromising the array performance.

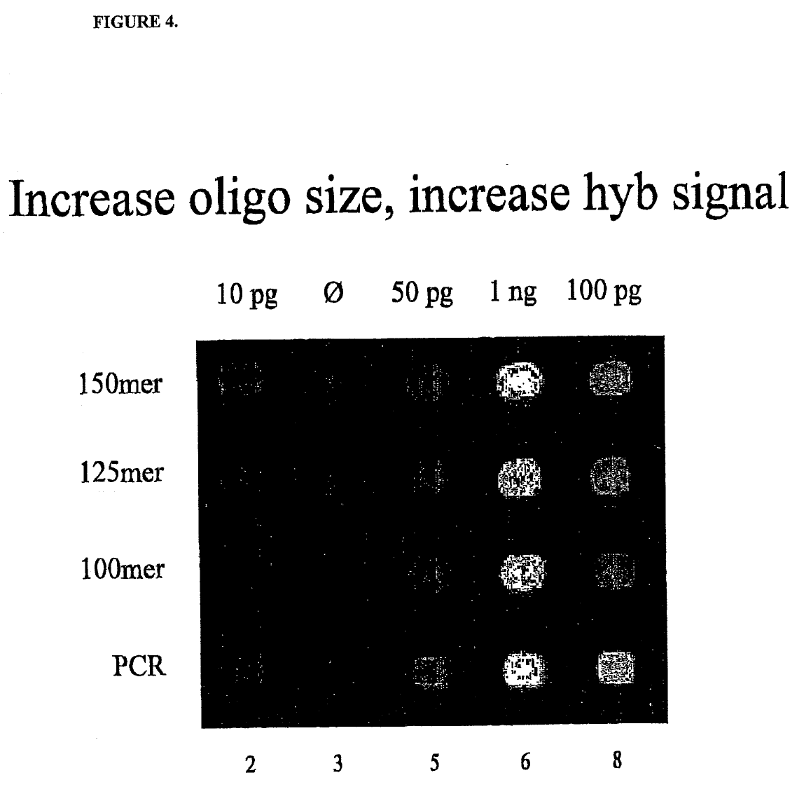

To test and benchmark the utility of non-modified long oligonucleotides as probes on DNA array fabricated on GAPS coated slide surface, a proof of principle experiment was conducted in a matrix to test for minimum oligonucleotide length able to detect 10 pg of cDNA and the overall hybridization signal after hybridization, as seen in FIG. 4. Three sets of oligonucleotides were printed with lengths of 150 bases, 125 bases, and 100 bases (all oligonucleotides were synthesized by Sigma-Genosys and purified by butanol extraction, also known as desalting). A set of PCR products was also printed as a positive control. Each oligonucleotide or PCR product represents one of five B. subtilis control genes (2, 3, 5, 6, and 8).

Oligonucleotides were printed at a concentration of 1 mg/mL in 3× sodium chloride sodium citrate (SSC) and PCR product were printed at 0.25 mg/mL in 3× SSC. Robotic printing was carried out using quill pins and coated slides. Slides were dried and stored in a vacuum desiccator at 650 mBar until ready for use. Slides were hybridized with Cy5 labeled cDNA, made from in vitro transcribed RNA. Control gene 2 was hybridized with 10 pg of the labeled cDNA in at 50 μL hybridization. As is seen, the 10 pg hybridization probe is detected on all the samples but most strongly on the 150-mer and PCR product spots. At higher amounts of other labeled cDNA, added to the hybridization mixture, the 150-mer and 125-mer performed favorably compared to PCR products when hydridized.

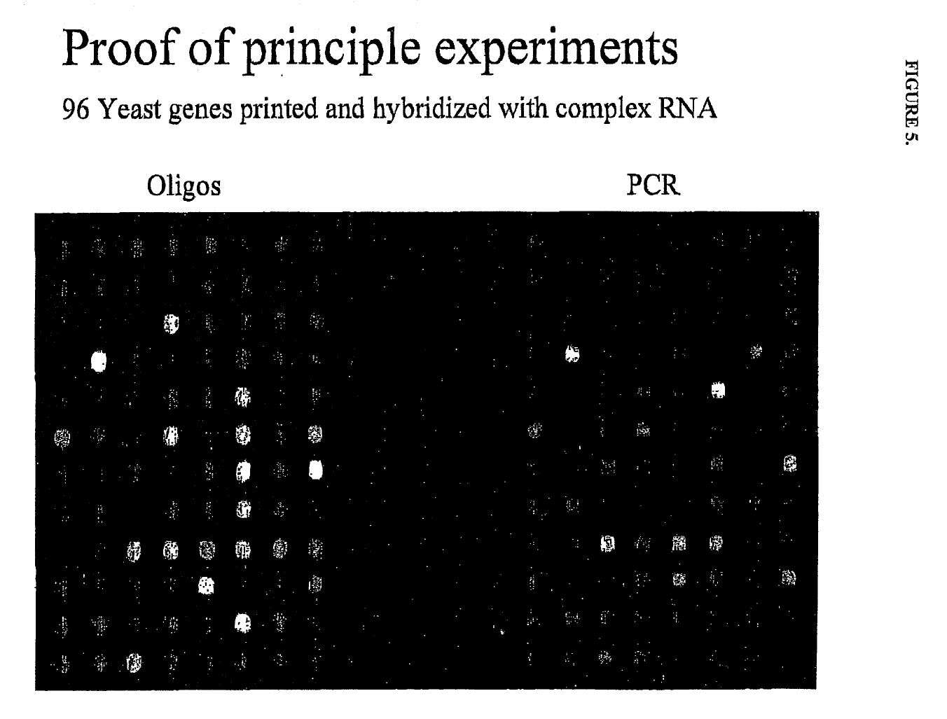

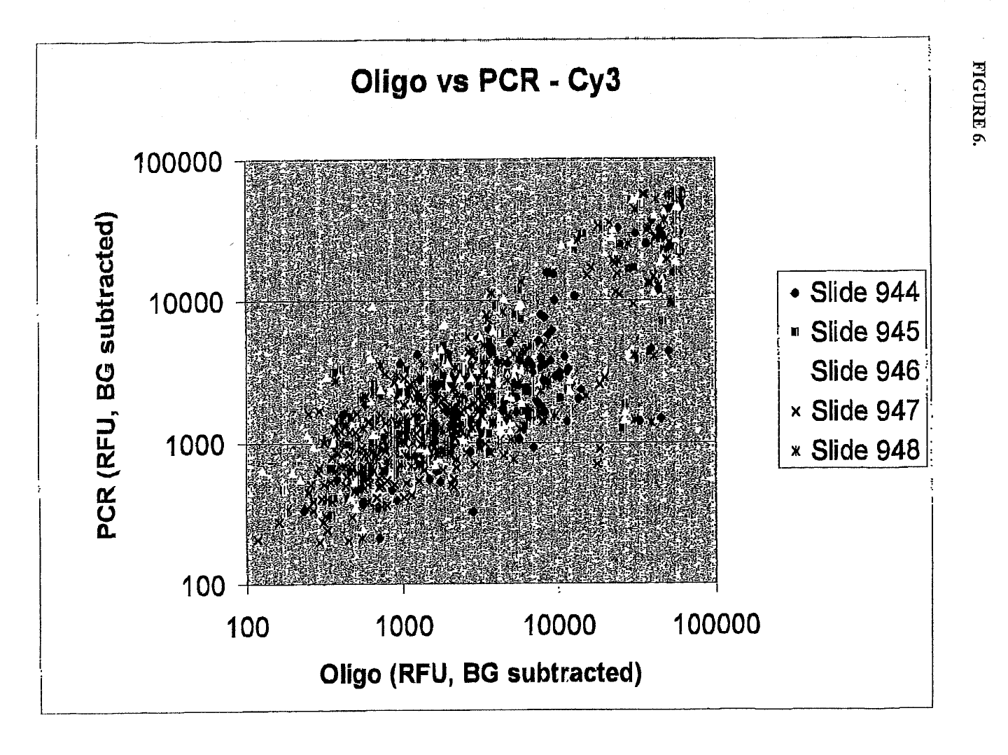

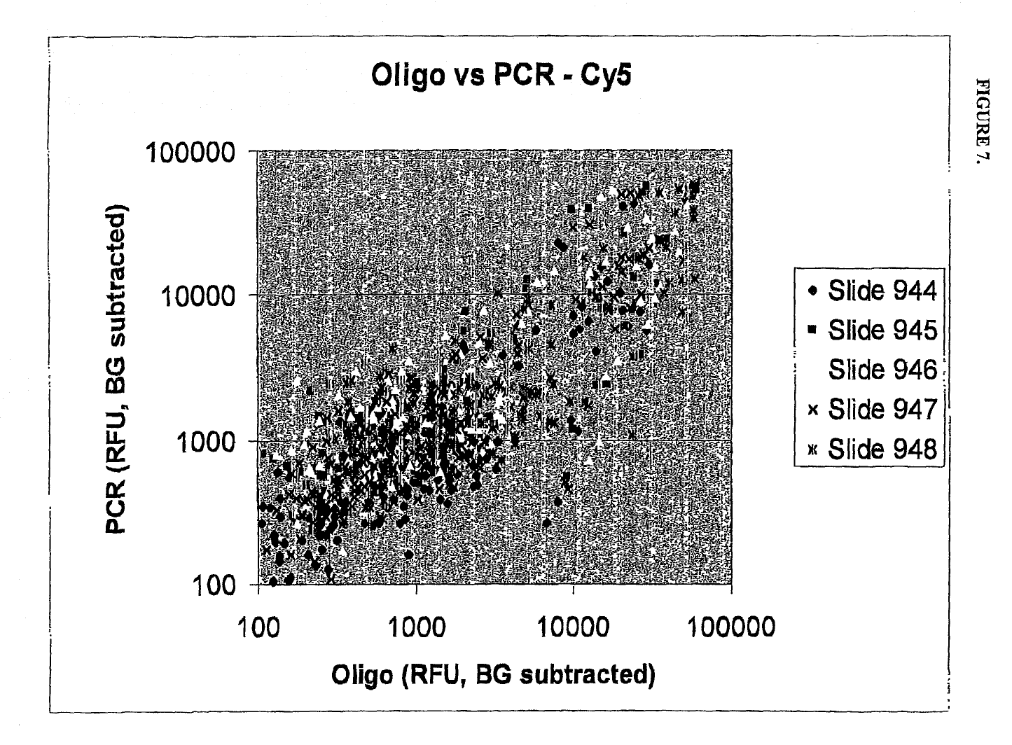

Relative fluorescence unit (RFU) values obtained from hybridization using 150-mer oligonucleotide spots were found to exhibit similar or equivalent results as PCR-product spots when a solution probe is a complex solution of cDNA labeled from yeast RNA. Ninety-six oligonucleotides and ninety-six PCR-products were printed on GAPS slides each representing a unique yeast gene, and stored as described above. The slides were hybridized to yeast cDNA generated from total RNA. FIG. 5 shows a representative result. The oligonucleotide biosites on the array appear to emit comparable, if not better results than the PCR-product, and with a stronger fluoresce in some cases. FIGS. 6 and 7 plot the extracted intensity values from each spot of five slides, where RFUs from the oligonucleotides are plotted along the x-axis and RFUs from the PCR-product are plotted along the y-axis. Most of the spots cluster along a 45-degree line, indicating a strong correlation between PCR-product hybridization and 150-mer-oligonucleotide hybridization.

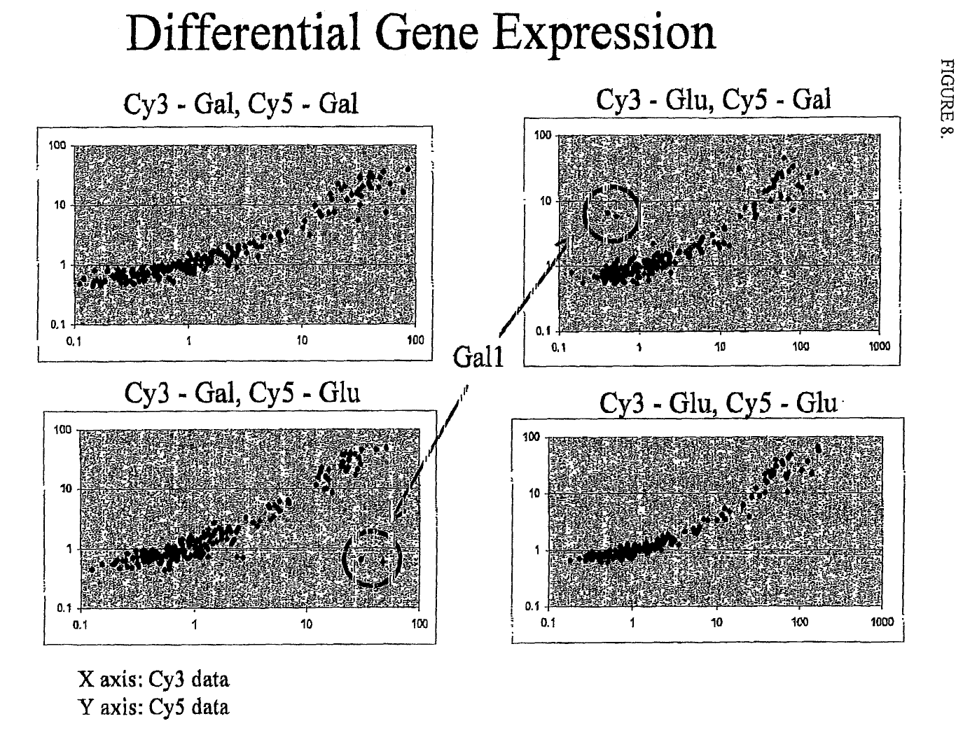

Since differential gene expression is the most common application for DNA arrays, the 150-mer oligonucleotides tested could detect a change in gene expression associated with a yeast cell's transition from galactose to glucose. The gene, Ga11, has been associated with the glucose to galactose transition for over a decade. For comparison purposes, FIG. 8 is a series of charts that plots RFU values from each of the four hybridizations. If a gene's relative level is unchanged it will be plotted near the 45-degree line; however, if there is a change it will appear in either the upper left or lower right corner of the graph. When glucose and galactose samples of cDNA are mixed together only the Ga11 gene (printed in duplicate) appears in the upper left or lower right comers. Remarkably, very few other spots deviate from the 45-degree line, thus long-length oligonucleotides appear to perform as well as PCR products. Total RNA was purified from cells grown continuously in galactose and cells grown in galactose for 12 hours and then switched to glucose for six hours. The RNA was labeled and mixed with Cyanine 3 and Cyanine 5 in all possible combinations and hybridized to the same kind of array with ninety-six oligonucleotides as described above.

Although the present invention has been described by way of examples, it will be understood by those skilled in the art that the invention is not limited to the embodiments specifically disclosed, and that various modifications and variations can be made without departing from the spirit and scope of the invention. Therefore, unless changes otherwise depart from the scope of the invention as defined by the following claims, they should be construed as included herein.

We claim:

1. An array for biological assays comprising a solid substrate having a plurality of biosites, each biosite having at least one set of chemically pre-synthesized, oligonucleotides of a length of about 30 bases or longer and attached to said substrate by predominantly non-covalent means.

2. The array according to claim 1, wherein said oligonucleotides exhibits no predetermined spatial orientation upon said substrate.

3. The array according to claim 1, wherein said oligonucleotides are unmodified.

4. The array according to claim 1, wherein said oligonucleotides have a length in a range from about a 30-mer to about a 80-mer.

5. The array according to claim 2, wherein said oligonucleotides have a length in a range from about a 50-mer to about a 75-mer.

6. The array according to claim 1, wherein said oligonucleotides have a length in a range from about a 80-mer to about a 180-mer.

7. The array according to claim 1, wherein said oligonucleotides are attached to a dendrimer.

8. The array according to claim 1, wherein said substrate has a surface made from a material selected from the following: plastics, glass, SiO₂, silanes, hydrogels, gold or platinum.

9. The array according to claim 1, wherein said substrate has a surface made from a material selected from the following: polyvinyl, polystyrene, polypropylene, polyester plastics.

10. The array according to claim 1, wherein said substrate has a surface made from glass.

11. An array for biological assays comprising a set of chemically pre-synthesized, single-stranded oligonucleotides, having a length of about 60 bases and longer, attached to multi-armed molecule that is in turn attached to a substrate by predominantly non-covalent means, wherein said oligonucleotides exhibits no predetermined spatial orientation upon said substrate.

12. The array according to claim 11, wherein said oligonucleotides have a length of in a range from about 70 bases to about 80 bases.

13. The array according to claim 11, wherein said oligonucleotides have a length of about 100 bases to about 150 bases.

14. The array according to claim 11, wherein said substrate has a surface made from a material selected from the following: plastics, glass, SiO₂, silanes, hydrogels, gold or platinum.

15. A method for fabricating microarrays, the method comprising: providing at least one chemically synthesized oligonucleotide having a length of about 30 bases and greater, affixing said oligoneucleotide to a substrate by a non-covalent means in a manner such that said oligonucleotide exhibits no predetermined orientation upon said substrate.

16. The method according to claim 15, wherein said oligonucleotide is not chemically modified.

17. The method according to claim 15, wherein said substrate possesses an electrostatic charge.

18. The method according to claim 15, wherein said oligonucleotide has a length of about 70 bases or greater and is affixed directly to said substrate.

19. The method according to claim 15, wherein said first oligonucleotide has a length of in a range from about 100 bases to about 150 bases.

20. The method according to claim 15, wherein said substrate has a surface made from a material selected from the following: plastics, glass, SiO₂, silanes, hydrogels, gold or platinum.
